(19) United States
(12) Patent Application Publication
(10) Pub. No.: US 2019/0046857 A1
(43) Pub. Date: Feb. 14, 2019

(54) REAL-TIME ATHLETIC PERFORMANCE DETECTION AND EVALUATION

(71) Applicant: Joe Sellers, III, Northville, MI (US)

(72) Inventor: Joe Sellers, III, Northville, MI (US)

(21) Appl. No.: 15/672,930

(22) Filed: Aug. 9, 2017

(51) Int. Cl. A63B 69/00; A61B 5/11; A61B 5/00; A63B 71/06

(52) U.S. Cl. CPC A63B 69/0026; A63B 71/10; A61B 5/6823; A61B 5/6824; A61B 5/6828; A61B 5/6829; A61B 5/486; A63B 71/0622; A63B 69/0024; A61B 2503/10; A63B 2071/0625; A63B 2071/0655; A63B 2220/836; A63B 2220/803; A63B 2225/50; A63B 2220/12; A61B 5/1112

(57) ABSTRACT

A system for evaluating an athlete's movement in real-time with respect to a predetermined target movement includes at least one sensor configured to detect a movement of the athlete's specific body part and generate a respective signal indicative of the detected movement. The system also includes a headset receiver configured to be worn by the athlete, in communication with the at least one sensor, configured to receive the respective signal from the sensor(s) and generate, in real-time, to the athlete a sensory feedback signal indicative of a quality of the detected movement. A method of evaluating an athlete's movement in real-time with respect to a predetermined target movement and using the above-described system is also disclosed.

### INTRODUCTION

The present disclosure relates to detection of an athlete's performance in real-time for effective evaluation and development thereof.

A physical sport or athletic competition generally requires a successful athlete to train his or her body both in dedicated drills and simulated game-time conditions. Such training is generally used by athletes to develop physical skills, body mechanics, as well as to improve stamina and strength, with a goal of enhancing athlete performance during game-time competition. Regular and consistent evaluation of an athlete's performance in real-time may be critical to identify specific areas for improvement and the athlete's overall performance development. However, real-time evaluation of an athlete's performance has generally been limited to observation and subjective evaluation by a coach or a trainer. Such subjective evaluation of an athlete's performance and body mechanics may make it difficult to objectively assess the athlete's performance, thereby extending and complicating the athlete's development process.

One example of a representative sport with an emphasis on effective body mechanics is hockey. Hockey may be played either on an ice or a dry rink using ice skates or roller blades, respectively. Hockey players typically work for years on perfecting their skating strides. The hockey stride involves numerous body motions, complex body mechanics, and forces that are difficult to replicate or adjust even with extensive coaching, training, and video analysis. Traditional training tools and methods typically depend on a hockey player taking training feedback and implementing the trainer's advice on the ice, both in practice and during the game. The majority of the time, skater development process relies on observing a video of the recorded skating stride off the rink, or talking to a skating coach after a skating drill has been performed.

### SUMMARY

A system for evaluating an athlete's movement in real-time with respect to a predetermined target movement includes at least one sensor configured to detect a movement of a particular body part of the athlete. The at least one sensor is also configured to generate a respective signal indicative of the detected movement. The system also includes a headset receiver configured to be worn by the athlete and in communication with the at least one sensor. The headset receiver is configured to receive the respective signal from the at least one sensor and generate, in real-time, to the athlete a sensory feedback signal indicative of a quality of the detected movement. The quality of the detected movement is defined by a comparison of the detected movement to the predetermined target movement.

The sensory feedback signal generated by the headset receiver may include at least one of an audible signal, a tactile signal, and a visual signal.

The sensory feedback signal may include a first feedback signal. In such a case, the headset receiver may be further configured to generate, in real-time, the first sensory feedback signal indicative of the detected movement failing to coincide with the predetermined target movement.

The sensory feedback signal may include a second feedback signal. The headset receiver may be further configured to generate, in real-time, the second sensory feedback signal indicative of the detected movement substantially coinciding with the predetermined target movement.

The system may additionally include a first data processing device, such as a personal computer, in communication with the at least one sensor. In such an embodiment, the first data processing device is configured to receive the respective signal from the at least one sensor, and programmed to process the received signal(s) and generate a user-readable output file indicative of the athlete's detected movement. The user-readable output file is intended to be used for evaluating and comparing, in real-time, the athlete's detected movement to the predetermined target movement to thereby facilitate athlete's peak performance.

The first data processing device may be further configured to generate a comparison of the athlete's detected movement to the predetermined target movement. Such a comparison may be, for example, either numeric or visual.

The system may also include a second data processing device, such as a cellular telephone, in communication with the at least one sensor. The second data processing device may be configured to receive the respective signals indicative of the detected movement from the at least one sensor. The second data processing device may be further programmed to process the received signals and display to a user of the subject device the received signals in a user-readable format to compare, in real-time, the athlete's detected movement to the predetermined target movement.

The at least one sensor may include a plurality of sensors, such as accelerometers. Such sensors may specifically include at least one of the following: a first sensor configured to be arranged on the athlete's shoulder, detect a movement of the athlete's shoulder, and communicate in, real-time, to at least one of the headset receiver, the first data processing device, and the second data processing device a signal indicative of the detected movement of the athlete's shoulder; a second sensor configured to be arranged on the athlete's wrist, detect a movement of the athlete's wrist, and communicate, in real-time, to at least one of the headset receiver, the first data processing device, and the second data processing device a signal indicative of the detected movement of the athlete's wrist; a third sensor configured to be arranged on the athlete's back, detect a movement of the athlete's back, and communicate, in real-time, to at least one of the headset receiver, the first data processing device, and the second data processing device a signal indicative of the detected movement of the athlete's back; a fourth sensor configured to be arranged on the athlete's hip, detect a movement of the athlete's hip, and communicate, in real-time, to at least one of the headset receiver, the first data processing device, and the second data processing device a signal indicative of the detected movement of the athlete's hip; a fifth sensor configured to be arranged on the athlete's knee, detect a movement of the athlete's fifth body part, and communicate, in real-time, to at least one of the headset receiver, the first data processing device, and the second data processing device a signal indicative of the detected movement of the athlete's knee; a sixth sensor configured to be arranged on the athlete's ankle, detect a movement of the athlete's ankle, and communicate, in real-time, to at least one of the headset receiver, the first data processing device, and the second data processing device a signal indicative of the detected movement of the athlete's ankle; and a seventh sensor configured to be arranged on the athlete's foot or skate, detect a movement of the athlete's foot, and communicate, in real-time, to at least one of the headset receiver, the first data processing device, and the second data processing device a signal indicative of the detected movement of the athlete's foot.

The at least one sensor may additionally include an eighth sensor in communication with a global positioning satellite (GPS). The eighth sensor may be configured to be arranged on the athlete's back to detect a skating speed of the athlete and communicate, in real-time, to at least one of the headset receiver, the first data processing device, and the second data processing device a signal indicative of the detected skating speed of the athlete.

The headset receiver may be incorporated into a helmet configured to be worn by the athlete.

The athlete may be a hockey player, and the movement may be a skating stride of such a player.

A method of evaluating an athlete's movement in real-time with respect to a predetermined target movement and using the above-described system is also disclosed.

The above features and advantages, and other features and advantages of the present disclosure, will be readily apparent from the following detailed description of the embodiment(s) and best mode(s) for carrying out the described disclosure when taken in connection with the accompanying drawings and appended claims.

### BRIEF DESCRIPTION OF THE DRAWINGS

FIG. 1 is an illustration of a representative athlete in full extension during a forward skating stride, and a system for evaluating the athlete's movement in real-time (having a plurality of sensors for detection of the movements and data processing devices for receiving sensor signals) according to the disclosure.

FIG. 2 is an illustration of the athlete and the system shown in FIG. 1, the forward skating stride depicted in full recovery according to the disclosure.

FIG. 3 is an illustration of the athlete and the system shown in FIG. 1, the athlete depicted performing a forward cross-over around a turn according to the disclosure.

FIG. 4 is an illustration of the athlete and the system shown in FIG. 1, the athlete depicted in full extension during a backward skating stride according to the disclosure.

FIG. 5 is an illustration of the athlete and the system shown in FIG. 4, the backward skating stride depicted in full recovery according to the disclosure.

FIG. 6 is an illustration of the athlete and the system shown in FIG. 4, the athlete depicted performing a backward cross-over around a turn according to the disclosure.

FIG. 7 is a flow chart illustrating a method of evaluating an athlete's movement in real-time with respect to a predetermined target movement according to the present disclosure.

### DETAILED DESCRIPTION

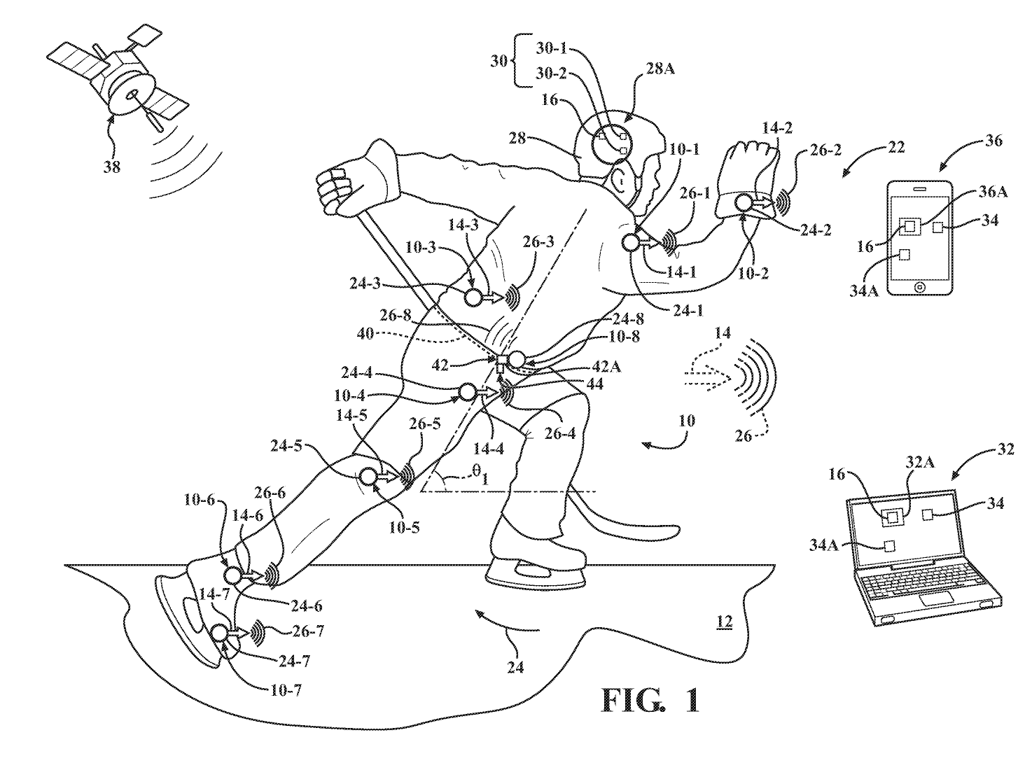

Referring to the drawings, wherein like reference numbers refer to like components, FIG. 1 shows a schematic view of an athlete 10 positioned relative to a playing or practice field surface 12. As shown, the field surface 12 may be an ice rink for playing ice hockey or a dry rink for playing roller or inline hockey. Although the remainder of the present disclosure will primarily concentrate on the athlete 10 being engaged in the sport of ice hockey, wherein the field surface 12 is an ice rink, nothing precludes the disclosure from being applicable to other physical sports or competition requiring training and practice to achieve peak athletic performance. Such various sports may, without limitation, include speed skating, lacrosse, soccer, American football, basketball, baseball, golf, etc. The hockey skater is discussed herein primarily as a representative athlete 10, due to the inherent mechanical complexity of the skating stride, to be described in detail below. The ensuing description of the skating stride is additionally intended to emphasize the applicability of the present disclosure to a variety of athletic skills, irrespective of the particular technique or the complexity of the underlying motion.

For effective and competitive performance during actual competition, physical sports generally require the athlete 10 to possess a series or an assortment of movements 14 that approach some theoretical target movements 16 having sound basis in physics. Specifically, the sport of hockey generally requires the athlete 10 to develop an effective skating stride that balances agility and control with stability and outright speed. A proper skating posture is the initial building block for an effective skating stride. The athlete's knees should be bent approximately 90 degrees. The knees should be over the athlete's toes, with proper ankle flex. The athlete's upper body should be tilted at an approximately 45 degree angle θ₁ relative to the rink surface, while the shoulders should be in line with knees and toes. Consistent with physics, speed during skating is generated from a push force that is applied to the rink surface perpendicular to the skate blade. This fact is true for skating at all speeds, both on dry and ice surfaces. When a skating athlete starts a forward stride from a dead stop, the skate blade is not moving. The athlete turns the skate approximately ninety degrees relative to the selected direction of movement across the surface and pushes off. Because initially there is no gliding motion, the acceleration is completely dependent on the stride length and the stride frequency.

The initial portion or phase of the stroke is predominantly and effectively an impulse pushing motion. Therefore, the more quickly the push is delivered, the faster the athlete will accelerate. Such a repetitive initial phase of the stroke may also be referred to as the sprint phase. As the athlete builds speed, the skate begins to glide through some part of the stroke. The gliding phase of the stroke permits the skate to exert pressure against the rink surface for a greater distance and a longer period of time. Because pressure to the rink surface may and should be delivered while the skate is gliding, such extended application of pressure generally results in greater acceleration of the athlete. The athlete's body extension during the stride is critical for an effective glide portion of the stroke. The stride leg should be extended to the side, not back. Pushing should be accomplished through the balls of the feet to maintain proper extension and increase the glide phase. The stride leg should be extended fully to build power and load the glide leg so the next push has as much power as may be possible to put into the rink surface. Because the impulse push of the sprint phase cannot deliver sustained acceleration at increasing speeds, the faster skaters in hockey have learned to apply pressure to the rink surface for a greater length of each stroke through the glide phase.

The gliding and the sprint phases of the stroke are inversely related with respect to their potential to deliver skater's acceleration. Specifically, as the skater's speed increases, of the potential to deliver acceleration of the gliding phase increases, while the potential to deliver acceleration via the sprint phase of the stroke decreases. As a result, at low skating speeds the sprint phase delivers more effective acceleration than the gliding phase, but at higher speeds the gliding portion of the stroke is necessary to generate acceleration beyond the acceleration developed by the sprint portion. While the amount of time spent gliding increases with athlete's speed, the application of the resulting push force shifts from the transverse plane toward a parallel plane relative to the selected direction of movement. Consequently, during the sprint phase of the stroke the skate blade generally finishes behind the skater's torso, while finishing more to the side of the skater's torso during the glide phase.

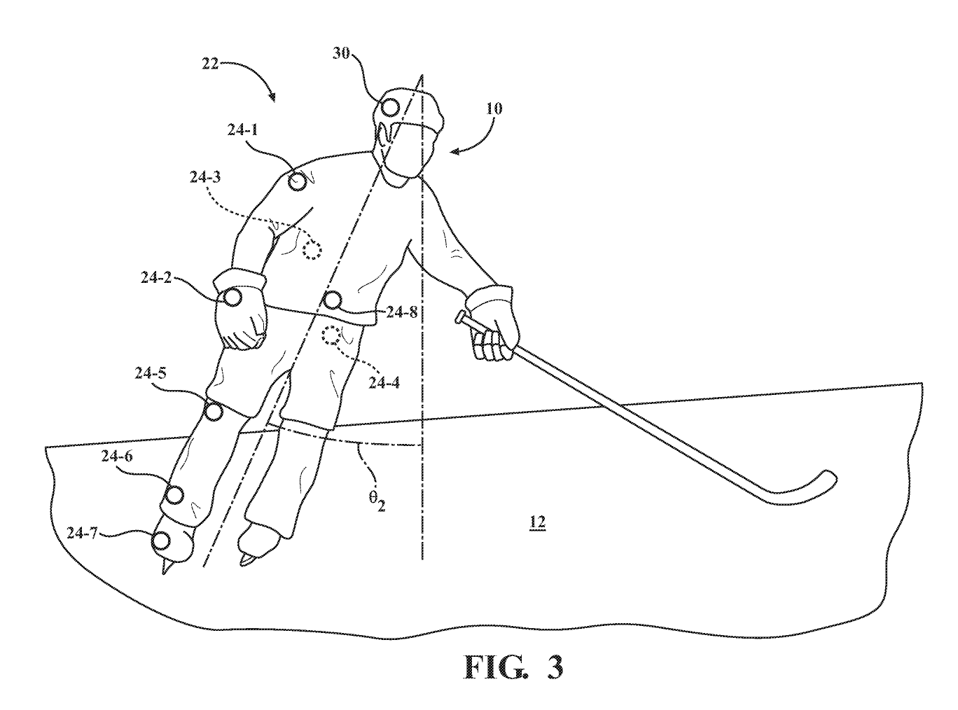

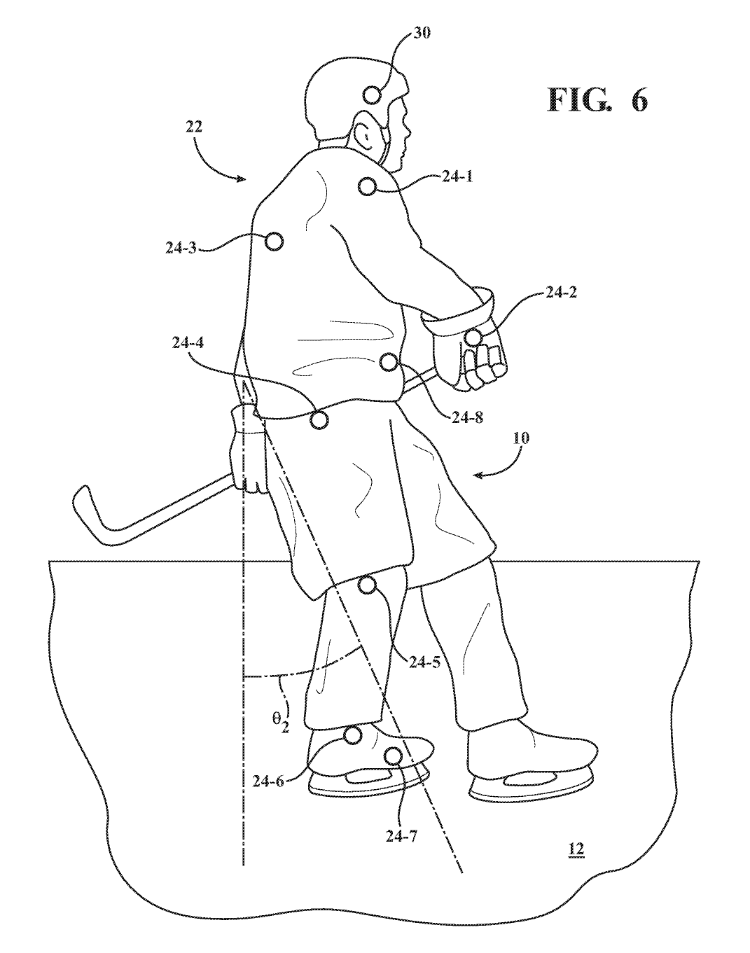

During a straight-line skating stroke, the skater's torso is situated substantially over or above the pushing skate. Additionally, a force vector, i.e., the amount and direction of the pushing force delivered by the skater into the rink surface, is vertical and perpendicular to the rink surface. However, when the skater is turning, because the athlete is angled into the turn, illustrated as angle θ₂ in FIGS. 3 and 6, while the force vector must still be delivered into the rink surface, the force vector becomes angled through the skater's torso and into the rink surface through the skate. Accordingly, the angle of the force vector changes with the position of the skater's torso relative to the skating surface (which is related to the tightness of the turn), the direction of skater's travel, and the skater's speed. The skating stroke may build speed during the glide phase if the athlete produces early and continuous pressure against the rink surface. To get early and continuous pressure to the rink surface, the athlete must recover the stride leg directly under the body, such that the torso is maintained over the force vector. With the skater's body weight over the force vector, the athlete may deliver maximum pressure down into the rink surface and control the glide phase.

For turning, skaters generally use the cross-over stride technique, where the athlete pushes off with the balls of the feet, and alternatively pushes with the outside and then the inside leg. Throughout the turn, proper ankle flex and knee bend are critical, while the athlete's head and hands lead into the turn and cross. Additionally, the knee and toe of the athlete's outside leg lead, and come across the body first. The athlete's hips should become involved in the stride when leaning into turns, while “grabbing” the rink surface with the inside leg when crossing over to make the turn. To complete the cross-over stride, full extension and the glide phase of the stroke should be executed with the inside leg.

Simply increasing the stroke count will not increase pressure during the glide phase. On the other hand, the glide phase, when weighted and timed properly, generates acceleration beyond the sprint portion of the stroke. To control the glide phase, the athlete must position the torso over the force vector into the rink surface at the start of each new stroke. If the athlete does not get the torso completely over the force vector, the skating stroke will be cut short. Specifically, as the skater quickly puts the skates on the rink surface, the skater's upper body does not transition over the force vector, and the glide phase is either short or entirely non-existent. Such a skating style typically results in quick stride recovery, substandard pressure to the rink surface, and a limited glide phase. Overall, a majority of hockey players at all levels could make improvements in their speed, power, and efficiency by improving their body position and optimizing duration and timing of the glide phase.

Additionally, throughout the stride, the athlete's arms should be swinging from side to side to help load the glide leg properly. By swinging from east to west instead of north to south, the athlete should be able to put more power and strength into the glide leg for the next push. The athlete's shoulders should remain square to the rink surface, the elbows should be slightly bent, and the athlete's head should be relatively static with respect to the torso. An effective backward skating stride differs from the forward stride in its specific mechanics, as well as in stride length. The backward stride generally uses “C-cuts” that primarily rely on the glide phase of the stroke, as described above with respect to the forward stride. As with the forward stride, proper athlete posture for backward stride is critical—with knees bent, pressure on balls of feet, shoulders back, and the athlete's head up. The C-cut should be a smooth push with full recovery of the skater's push-off knee back under the athlete's body. By using the upper body properly, the athlete should be able to get more power and speed out of each stride. The athlete's hips should also become involved in the stride when leaning into turns, while “gripping” the rink surface with the inside leg as the outside leg crosses over to make the turn.

According to the present disclosure, a system 22 is employed for evaluating, in real-time, a specified movement, generally indicated via numeral 14, of the athlete 10 with respect to a predetermined target movement 16. The underlying operation of the system 22 includes real-time collection of performance indicators for the athlete 10 facilitating real-time, as well as subsequent evaluation of the movement 14 with respect to the predetermined target movement 16. The athlete 10 may be a hockey player, and the movement 14 may be any of the movements for any individual body parts necessary for affecting the skating stride, the for either inline or ice hockey, as described above. As also noted above, the athlete 10 may be a player in a different sport which requires specific movement(s) to be practiced and trained for achieving desired movement(s), such as the target movement 16, for effective game-time performance.

The system 22 includes one or more sensors 24, for example, accelerometers, configured to detect the movement 14 of a particular body part of the athlete 10 and generate a respective signal, generally indicated via numeral 26, indicative of thus detected movement. The system 22 also includes a headset receiver 28 configured to be worn by the athlete 10. The headset receiver is in communication with the sensor(s) 24, and is configured to receive the respective signal 26. Additionally, the headset receiver 28 is configured to generate to the athlete 10, in real-time, a readily perceived sensory feedback signal 30 indicative of a quality of the detected movement 14. A measure of the quality of the detected movement 14 is specifically intended to be defined by a comparison of the detected movement to the predetermined target movement 16. The headset receiver 28 may be incorporated into a helmet 28A configured to be worn by the athlete 10. The sensory feedback signal 30 generated by the headset receiver 28 may be either an audible signal, such as an instantly recognizable sound, a tactile signal, such as a vibration of appropriate intensity, or a visual signal, such as a constantly shining or blinking light.

The sensory feedback signal 30 may include a first feedback signal 30-1 and a distinct second feedback signal 30-2. Additionally, the predetermined target movement 16 may be pre-programmed into the headset receiver 28. Furthermore, the headset receiver 28 may then be configured to generate, in real-time, the first sensory feedback signal 30-1 for perception by the athlete 10 when the detected movement 14 fails to coincide, within a predetermined allowable tolerance band or variation, with the predetermined target movement 16. Thus, the first feedback signal 30-1 may be used to alert the athlete 10 when the detected movement 14 does not match the target movement 16, for example a full stride recovery, as required for the specific hockey stride. Additionally, the headset receiver 28 may be configured to generate, in real-time, the second sensory feedback signal 30-2 for perception by the athlete 10 when the detected movement 14 substantially coincides with the predetermined target movement 16. Thus, the second feedback signal 30-2 may be used to inform the athlete 10 when the detected movement 14 substantially matches the target movement 16, for example, for every time the athlete achieves a full stride recovery, as required for the specific hockey stride. Accordingly, a measure of the quality of the detected movement 14 may be how much, within some predetermined allowable range of movement, the detected movement actually diverges from the predetermined target movement 16. The first feedback signal 30-1 and the second feedback signal 30-2 may be distinguished by individual types of signals, e.g., audible, tactile, or visual, or by quality of the first and second signals, e.g., intensity or color.

The system 22 may also include a first data processing device 32, such as a laptop or a personal computer, in communication with sensor(s) 24. The first data processing device 32 is configured to receive, wirelessly, from the sensor(s) 24 the respective signal 26 indicative of the detected movement 14. The first data processing device 32 is programmed to process the received signal(s) 26 and generate a user-readable output file 34 indicative of the athlete's detected movement 14. The output file 34 may be used either by the athlete 10, the athlete's coach or trainer for evaluating and comparing, in real-time, the athlete's detected movement 14 to the predetermined target movement 16, which may then be used to facilitate improvement in athlete's performance. Additionally, the predetermined target movement 16 may be pre-programmed into the first data processing device 32. The first data processing device 32 may be further configured or programmed to generate a comparison 34A of the athlete's detected movement 14 to the predetermined target movement 16. A representative comparison 34A may, for example, be in the form of a numeric, or a visual comparison or assessment, such as using a bar graph analysis. Overall, the comparison 34 is intended to clarify and/or emphasize the difference or concurrence between the detected movement 14 and the predetermined target movement 16.

It is intended that the first data processing device 32 includes a memory 32A, at least some of which is tangible and non-transitory. The memory 32A may be any recordable medium that participates in providing computer-readable data or process instructions. Such a medium may take many forms, including but not limited to non-volatile media and volatile media. Non-volatile media for the first data processing device 32 may include, for example, optical or magnetic disks and other persistent memory. Volatile media may include, for example, dynamic random access memory (DRAM), which may constitute a main memory. Such instructions may be transmitted by one or more transmission medium, including coaxial cables, copper wire and fiber optics, including the wires that comprise a system bus coupled to a processor of a computer.

Memory 32A of the first data processing device 32 may also include a floppy disk, a flexible disk, hard disk, magnetic tape, any other magnetic medium, a CD-ROM, DVD, any other optical medium, etc. The first data processing device 32 may be configured or equipped with other required computer hardware, such as a high-speed clock, requisite Analog-to-Digital (A/D) and/or Digital-to-Analog (D/A) circuitry, any necessary input/output circuitry and devices (I/O), as well as appropriate signal conditioning and/or buffer circuitry. Any algorithms required by the first data processing device 32 or accessible thereby may be stored in the memory and automatically executed to provide the required functionality.

The system 22 may additionally include a second data processing device 36 in communication with the sensor(s) 24. It is intended that the second data processing device 36 includes a memory 36A, which may be similar to the memory 32A described with respect to the first data processing device 32. The predetermined target movement 16 may be pre-programmed into the second data processing device 36. The second data processing device 36 may also be configured to receive, wirelessly, the respective signal(s) 26 from the sensor(s) 24 and programmed to process the received signals. The second data processing device 36 may be additionally configured to visually display to a user of the subject device the received signal(s) 26 in a user-readable format for evaluating, in real-time, the athlete's detected movement 14 relative to the predetermined target movement 16. The second data processing device 36 may, for example, be a cellular telephone or a hand-held tablet. The second data processing device 36 may be in electronic communication with the sensor(s) 24 via an appropriate wireless communication technology, such as the Bluetooth. The second data processing device 36 may be further configured or programmed to generate the comparison 34A of the athlete's detected movement 14 to the predetermined target movement 16. As described above with respect to the first data processing device 32, the comparison 34A may be in the form of a numeric, or a visual comparison, such as using a bar graph analysis.

With respect to the athlete 10 situated as the above-described hockey player, the one or more sensors 24 may include a plurality of sensors, each configured to detect a specific movement of the skater's particular body part. More particularly, the plurality of sensors 24 may include a first sensor 24-1 configured, i.e., designed and constructed, to be arranged on the athlete's shoulder 10-1. The first sensor 24-1 is accordingly configured to detect a movement 14-1 of the athlete's shoulder 10-1 and communicate, in real-time, to either one or all of the headset receiver 28, the first data processing device 32, and the second data processing device 36 a signal 26-1 indicative of the detected movement 14-1. The plurality of sensors 24 may also include a second sensor 24-2 configured to be arranged on the athlete's wrist 10-2. The second sensor 24-2 is configured to detect a movement 14-2 of the athlete's wrist 10-2 and communicate, in real-time, to either one or all of the headset receiver 28, the first data processing device 32, and the second data processing device 36 a signal 26-2 indicative of the detected movement 14-2.

The plurality of sensors 24 may also include a third sensor 24-3 configured to be arranged on the athlete's back 10-3. The third sensor 24-3 is configured to detect a movement 14-3 of the athlete's back 10-3 and communicate, in real-time, to either one or all of the headset receiver 28, the first data processing device 32, and the second data processing device 36 a signal 26-3 indicative of the subject detected movement. The plurality of sensors 24 may additionally include a fourth sensor 26-4 configured to be arranged on the athlete's hip 10-4. The fourth sensor 24-4 is configured to detect a movement 14-4 of the athlete's hip 10-4 and communicate, in real-time, to either one or all of the headset receiver 28, the first data processing device 32, and the second data processing device 36 a signal 26-4 indicative of the subject detected movement.

The plurality of sensors 24 may also include a fifth sensor 24-5 configured to be arranged on the athlete's knee 10-5. The fifth sensor 24-5 is configured to detect a movement 14-5 of the athlete's knee 10-5 and communicate, in real-time, to either one or all of the headset receiver 28, the first data processing device 32, and the second data processing device 36 a signal 26-5 indicative of the subject detected movement. The plurality of sensors 24 may additionally include a sixth sensor 24-6 configured to be arranged on the athlete's ankle 10-6. The sixth sensor 24-6 is configured to detect a movement 14-6 of the athlete's ankle 10-6 and communicate, in real-time, to either one or all of the headset receiver 28, the first data processing device 32, and the second data processing device 36 a signal 26-6 indicative of the subject detected movement.

The plurality of sensors 24 may also include a seventh sensor 24-7 configured to be arranged on the athlete's foot 10-7, or on a skate worn by the athlete 10. The seventh sensor 24-7 is configured to detect a movement 14-7 of the athlete's foot 10-7 and communicate, in real-time, to either one or all of the headset receiver 28, the first data processing device 32, and the second data processing device 36 a signal 26-7 indicative of the subject detected movement. Furthermore, the plurality of sensors 24 may additionally include an eighth sensor 24-8 in communication with a global positioning satellite (GPS) 38. As shown, the eighth sensor 24-8 is configured to be arranged on the athlete's waist 10-8, such as on a detachable waste band 40, to detect a skating speed of the athlete 10. The eighth sensor 24-8 is additionally configured to communicate, in real-time, to either one or all of the headset receiver 28, the first data processing device 32, and the second data processing device 36 a signal 26-8 indicative of the athlete's detected skating speed. The athlete's waist 10-8 is deemed to be an appropriate location for the eighth sensor 24-8 because the skater's waist is generally coincident with the middle of skater's body, and may be used to define the athlete's position at any moment in time relative to the rink surface 12. In addition to the speed of the player, the eighth sensor 24-8 may facilitate a determination of the number of skating strides per a specified distance.

The system 22 may also include a hub 42 having a signal processing unit 42A in communication with the GPS 38 and configured to centrally gather the signals 26-1 through 26-8 from the sensors 24-1 through 24-8 and transmit via Bluetooth or WiFi to each of the headset receiver 28, the first data processing device 32, and the second data processing device 36. The hub unit 42 may be incorporated into the waist band 40. The hub 42 may include a switch 44 configured to change a mode programmed into the signal processing unit 42A from forward stride to backward stride to detect and provide appropriate feedback with respect to the stride mechanics being detected. Alternatively, the signal processing unit 42A may be programmed to recognize using the signals 26-5 through 26-7 received from some or all of the sensors 24-5 thorough 24-7, such as via the GPS 38, whether the athlete 10 is skating backward or forward and provide appropriate feedback to the athlete with respect to the corresponding mechanics via the sensory feedback signal 30.

Each of the first data processing device 32 and the second data processing device 36 may include a downloaded application program tailored for the specific subject device for appropriately processing and displaying the received signals 26-1 through 26-8. Furthermore, the predetermined target movement 16 programmed into each of the headset receiver 28, into the memory 32A of the first data processing device 32, and into the memory 36A of the second data processing device 36 may include individual movement targets corresponding to the above signals 26-1 through 26-8 to support evaluation of each of the subject signals.

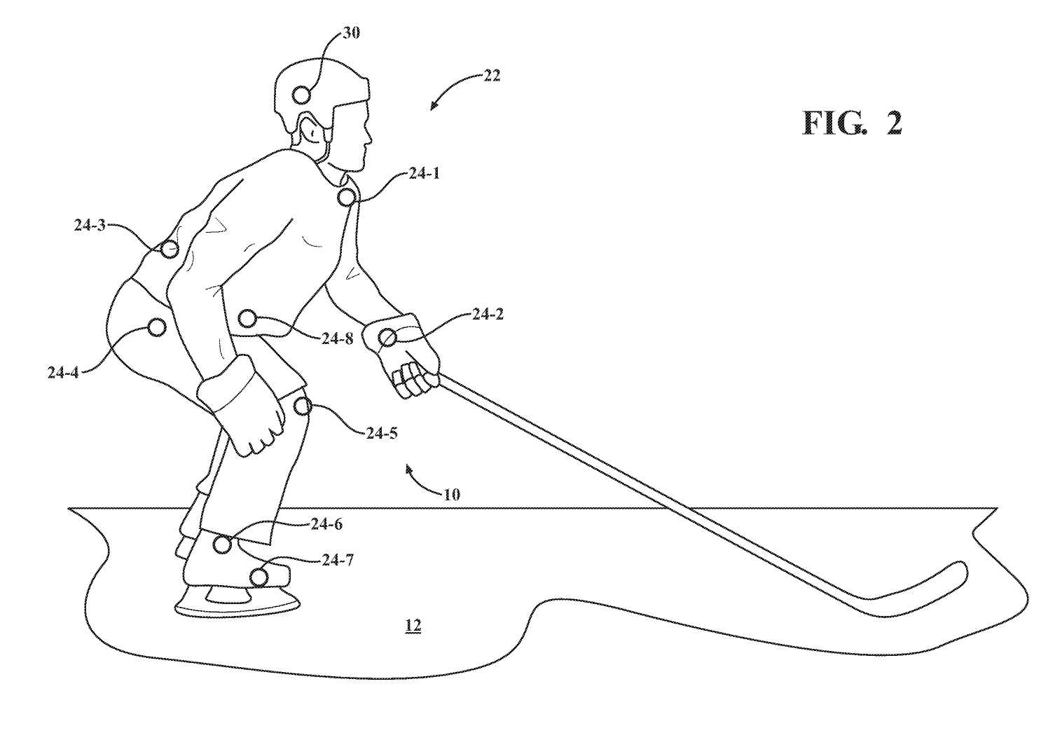

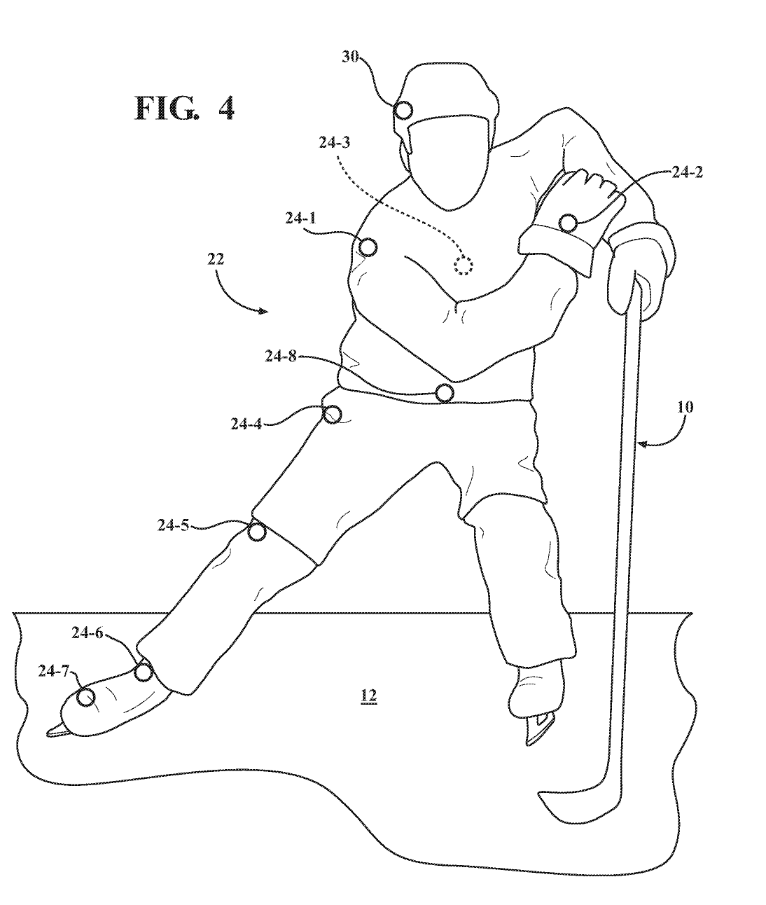

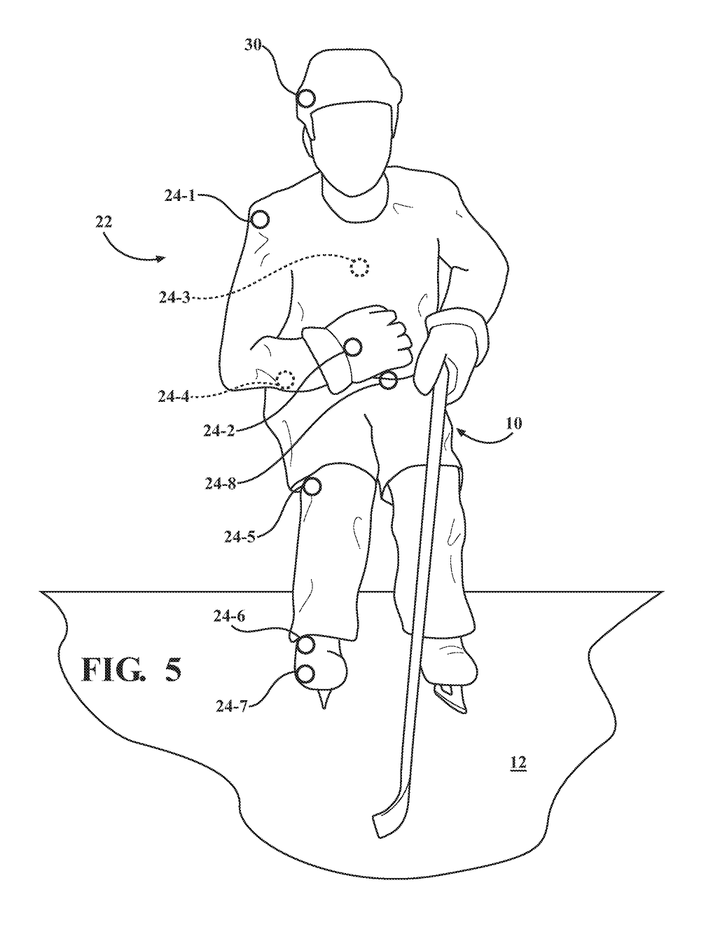

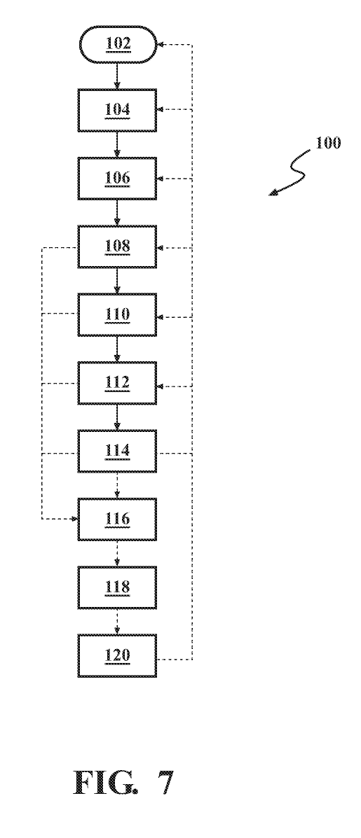

FIG. 7 depicts a method 100 of collecting performance indicators of the athlete 10, such as the hockey skater, and evaluating the athlete's movement 14 in real-time with respect to the predetermined target movement 16, as described above with respect to FIGS. 1-6. The method 100 commences in frame 102 with detecting, via at least one sensor 24, such as the sensors 24-1 through 24-8, the movement 14 of a particular body part, such as the shoulder 10-1, athlete's wrist 10-2, athlete's back 10-3, the athlete's hip 10-4, athlete's knee 10-5, athlete's ankle 10-6, athlete's skate or foot 10-7, and athlete's waist 10-8. After frame 102, the method advances to frame 104, where it includes generating, via the sensor(s) 24, the respective signal(s) 26, such as the signals 26-1 through 26-8, indicative of the detected movement 14, such as the specific movements 14-1 through 14-8. Following frame 104, the method proceeds to frame 106, where the method includes receiving, via the headset receiver 28 worn by the athlete 10 and in communication with the sensor(s) 24, the respective signal(s) 26 from the subject sensor(s) 24.

After frame 106, the method advances to frame 108, where it includes generating, in real-time, to the athlete 10, via the headset receiver 28, the sensory feedback signal 30 indicative of the quality of the detected movement 14. As described above with respect to FIGS. 1-6, the sensory feedback signal 30 may include the first feedback signal 30-1. In such an embodiment, in frame 108 the method may include generating, via the headset receiver 28, in real-time, the first sensory feedback 30-1 signal to the athlete 10 indicative of the detected movement 14 failing to coincide with the predetermined target movement 16. As also described above, the sensory feedback signal 30 may include the second feedback signal 30-2. In the subject embodiment, in frame 108 the method may include generating, via the headset receiver 28, in real-time, the second sensory feedback signal 30-2 to the athlete 10 indicative of the detected movement coinciding with the predetermined target movement 16.

Following frame 108, the method advances to frame 110, where it includes receiving, via the first data processing device 32 in communication with the sensor(s) 24, the respective signal(s) 26. Following frame 110, the method proceeds to frame 112, where the method includes processing, via the first data processing device 32, the received signal(s) 26 and generating the user-readable output file 34 indicative of the athlete's detected movement 14. In frame 112, the method may also include generating, via the first data processing device 32, the comparison 34A of the athlete's detected movement 14 to the predetermined target movement 16. After frame 112, the method advances to frame 114. In frame 114, the method includes comparing, in real-time, the user-readable output file 34 to the predetermined target movement 16 of the specific body part, such as the shoulder 10-1, wrist 10-2, back 10-3, hip 10-4, knee 10-5, ankle 10-6, foot 10-7, or waist 10-8. The comparison 34A of the athlete's detected movement 14 to the predetermined target movement 16 may also be performed via the second data processing device 36 specifically programmed to generate the subject comparison.

Following any of the frames 108-114, as described above with respect to FIGS. 1-6, the method may proceed to frame 116 where the method includes receiving, via the second data processing device 36, the respective signal(s) 26 from the respective sensor(s) 24. After frame 116, the method may continue on to frame 118. In frame 118, the method includes processing the received signal(s) 26 and displaying to the user of the second data processing device 36 the received signal(s) in a user-readable format. Following frame 118, the method may proceed to frame 120, where the method includes comparing, in real-time, via the second data processing device 36, the athlete's detected movement 14 to the predetermined target movement 16.

Following either frame 114 or frame 120, the method may loop back to any of the frames 102-112 to receive and reassess the signal(s) 26 from any of the appropriate sensor(s) 24 to determine and determine an updated position of any of the constituent body parts. Overall, real-time detection and communication of the athlete's movement(s) 14 by the system 22 and the method 100 facilitates real-time analysis of the movement and facilitate quicker and otherwise more effective athlete development and achievement of the athlete's peak performance.

The detailed description and the drawings or figures are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims. Furthermore, the embodiments shown in the drawings or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

What is claimed is:

1. A system for evaluating an athlete's movement in real-time with respect to a predetermined target movement, the system comprising:
at least one sensor configured to detect a movement of a particular body part of the athlete and generate a respective signal indicative of the detected movement; and
a headset receiver configured to be worn by the athlete, in communication with the at least one sensor, configured to receive the respective signal from the at least one sensor and generate, in real-time, to the athlete a sensory feedback signal indicative of a quality of the detected movement, wherein the quality of the detected movement is defined by a comparison of the detected movement to the predetermined target movement.

2. The system according to claim 1, wherein the sensory feedback signal generated by the headset receiver includes at least one of an audible signal, a tactile signal, and a visual signal.

3. The system according to claim 2, wherein the sensory feedback signal includes a first feedback signal, and wherein the headset receiver is further configured to generate, in real-time, the first sensory feedback signal indicative of the detected movement failing to coincide with the predetermined target movement.

4. The system according to claim 3, wherein the sensory feedback signal includes a second feedback signal, and wherein the headset receiver is further configured to generate, in real-time, the second sensory feedback signal indicative of the detected movement coinciding with the predetermined target movement.

5. The system according to claim 1, further comprising a first data processing device in communication with the at least one sensor, configured to receive the respective signal from the at least one sensor, and programmed to process the received signal and generate a user-readable output file indicative of the athlete's detected movement for comparing, in real-time, the athlete's detected movement relative to the predetermined target movement.

6. The system according to claim 5, wherein the first data processing device is further configured to generate a comparison of the athlete's detected movement to the predetermined target movement.

7. The system according to claim 5, further comprising a second data processing device in communication with the at least one sensor, configured to receive the respective signals from the at least one sensor, and programmed to process the received signals and display to a user of the second data processing device the received signals in a user-readable format to compare, in real-time, the athlete's detected movement to the predetermined target movement.

8. The system according to claim 7, wherein the at least one sensor includes a plurality of sensors, including at least one of:
a first sensor configured to be arranged on the athlete's shoulder, detect a movement of the athlete's shoulder, and communicate, in real-time, to at least one of the headset receiver, the first data processing device, and the second data processing device a signal indicative of the detected movement of the athlete's shoulder;
a second sensor configured to be arranged on the athlete's wrist, detect a movement of the athlete's wrist, and communicate, in real-time, to at least one of the headset receiver, the first data processing device, and the second data processing device a signal indicative of the detected movement of the athlete's wrist;
a third sensor configured to be arranged on the athlete's back, detect a movement of the athlete's back and communicate, in real-time, to at least one of the headset receiver, the first data processing device, and the second data processing device a signal indicative of the detected movement of the athlete's back;
a fourth sensor configured to be arranged on the athlete's hip, detect a movement of the athlete's hip, and communicate, in real-time, to at least one of the headset receiver, the first data processing device, and the second data processing device a signal indicative of the detected movement of the athlete's hip;
a fifth sensor configured to be arranged on the athlete's knee, detect a movement of the athlete's knee, and communicate, in real-time, to at least one of the headset receiver, the first data processing device, and the second data processing device a signal indicative of the detected movement of the athlete's knee;
a sixth sensor configured to be arranged on the athlete's ankle, detect a movement of the athlete's ankle, and communicate, in real-time, to at least one of the headset receiver, the first data processing device, and the second data processing device a signal indicative of the detected movement of the athlete's ankle; and
a seventh sensor configured to be arranged on the athlete's skate or foot, detect a movement of the athlete's foot, and communicate, in real-time, to at least one of the headset receiver, the first data processing device, and the second data processing device a signal indicative of the detected movement of the athlete's foot.

9. The system according to claim 8, wherein the at least one sensor additionally includes an eighth sensor in communication with a global positioning satellite (GPS), and configured to be arranged on the athlete's waist to detect a skating speed of the athlete and communicate, in real-time, to at least one of the headset receiver, the first data processing device, and the second data processing device a signal indicative of the detected skating speed of the athlete.

10. The system according to claim 1, wherein the headset receiver is incorporated into a helmet configured to be worn by the athlete.

11. The system according to claim 1, wherein the athlete is a hockey player and the movement is a skating stride.

12. A method of evaluating an athlete's movement in real-time with respect to a predetermined target movement, the method comprising:
detecting, via at least one sensor, a movement of a particular body part of the athlete;
generating, via the at least one sensor, a respective signal indicative of a quality of the detected movement, wherein the quality of the detected movement is defined by a comparison of the detected movement to the predetermined target movement;
receiving, via a headset receiver configured to be worn by the athlete and in communication with the at least one sensor, the respective signal from the at least one sensor; and
generating, in real-time, to the athlete, via the headset receiver, a sensory feedback signal indicative of the detected movement.

13. The method according to claim 12, wherein the sensory feedback signal generated by the headset receiver includes at least one of an audible signal, a tactile signal, and a visual signal.

14. The method according to claim 13, wherein the sensory feedback signal includes a first feedback signal, further comprising generating, via the headset receiver, in real-time, the first sensory feedback signal indicative of the detected movement failing to coincide with the predetermined target movement.

15. The method according to claim 14, wherein the sensory feedback signal includes a second feedback signal, further comprising generating, via the headset receiver, in real-time, the second sensory feedback signal indicative of the detected movement coinciding with the predetermined target movement.

16. The method according to claim 11, further comprising:
receiving, via a first data processing device in communication with the at least one sensor, the respective signal from the at least one sensor;
processing, via a first data processing device, the received signal and generating a user-readable output file indicative of the athlete's detected movement; and
comparing, in real-time, the user-readable output file relative to the predetermined target movement.

17. The method according to claim 16, further comprising generating, via the first data processing device, a comparison of the athlete's detected movement to the predetermined target movement.

18. The method according to claim 16, further comprising:
receiving, via a second data processing device in communication with the at least one sensor, the respective signals from the at least one sensor;
processing, via the second data processing device, the received signals;
displaying, via the second data processing device, to a user of the second data processing device the received signals in a user-readable format; and
comparing, via the second data processing device, in real-time, the athlete's detected movement to the predetermined target movement.

19. The method according to claim 18, wherein the at least one sensor includes a plurality of sensors having at least one of:
a first sensor arranged on the athlete's shoulder;
a second sensor arranged on the athlete's wrist;
a third sensor arranged on the athlete's back;
a fourth sensor arranged on the athlete's hip;
a fifth sensor arranged on the athlete's knee;
a sixth sensor arranged on the athlete's ankle; and
a seventh sensor arranged on the athlete's skate or foot;
the method further comprising at least one of:
detecting, via the first sensor, a movement of the athlete's shoulder and communicating, in real-time, to at least one of the headset receiver, the first data processing device, and the second data processing device a signal indicative of the detected movement of the athlete's shoulder;
detecting, via the second sensor, a movement of the athlete's wrist second body part and communicating, in real-time, to at least one of the headset receiver, the first data processing device, and the second data processing device a signal indicative of the detected movement of the athlete's wrist;
detecting, via the third sensor, a movement of the athlete's back and communicating, in real-time, to at least one of the headset receiver, the first data processing device, and the second data processing device a signal indicative of the detected movement of the athlete's back;
detecting, via the fourth sensor, a movement of the athlete's hip and communicating, in real-time, to at least one of the headset receiver, the first data processing device, and the second data processing device a signal indicative of the detected movement of the athlete's hip;
detecting, via the fifth sensor, a movement of the athlete's knee and communicating, in real-time, to at least one of the headset receiver, the first data processing device, and the second data processing device a signal indicative of the detected movement of the athlete's knee;
detecting, via the sixth sensor, a movement of the athlete's ankle and communicating, in real-time, to at least one of the headset receiver, the first data processing device, and the second data processing device a signal indicative of the detected movement of the athlete's ankle; and
detecting, via the seventh sensor, a movement of the athlete's foot, and communicating, in real-time, to at least one of the headset receiver, the first data processing device, and the second data processing device a signal indicative of the detected movement of the athlete's foot.

20. The method according to claim 19, wherein the at least one sensor additionally includes an eighth sensor arranged on the athlete's waist and in communication with a global positioning satellite (GPS), further comprising detecting, via the eighth sensor, a skating speed of the athlete and communicating, in real-time, to at least one of the headset receiver, the first data processing device, and the second data processing device a signal indicative of the detected skating speed of the athlete.

21. The method according to claim 12, wherein the headset receiver is incorporated into a helmet configured to be worn by the athlete.

22. The method according to claim 12, wherein the athlete is a hockey player and the movement is a skating stride.
